(19) United States
(12) Patent Application Publication
(10) Pub. No.: US 2026/0182893 A1
(43) Pub. Date: Jul. 2, 2026

(54) Optical Sensor Integration into Chest Patch

(71) Applicant: iRhythm Technologies, Inc., San Francisco, CA (US)

(72) Inventors: Monica C. Lin, San Francisco, CA (US); Jeffrey Abercrombie, Oakland, CA (US); Ahmad Saleh, San Jose, CA (US)

(73) Assignee: iRhythm Technologies, Inc., San Francisco, CA (US)

(21) Appl. No.: 19/436,743

(22) Filed: Dec. 30, 2025

Related U.S. Application Data
(60) Provisional application No. 63/740,262, filed on Dec. 30, 2024.

(51) Int. Cl. A61B 5/333; A61B 5/00; A61B 5/024; A61B 5/08; A61B 5/1455; A61B 5/257; A61B 5/282

(52) U.S. Cl. CPC A61B 5/333; A61B 5/257; A61B 5/282; A61B 5/6823; A61B 5/6831; A61B 5/6833; A61B 5/02427; A61B 5/0803; A61B 5/0816; A61B 5/14552; A61B 2562/0219; A61B 2562/0233; A61B 2562/0271; A61B 2562/146; A61B 2562/164; A61B 2562/227

(57) ABSTRACT

Optical sensor integration into a chest patch is described. A wearable device comprises a housing configured to be attached to a chest region of an individual via an attachment component, the attachment component configured to conform to contours of the chest region, and an optical sensor configured to detect a PPG signal from the chest region of the individual. Various form factors are considered, including, but not limited to, a cartridge design, a wing design, a snap-in design, and/or an auxiliary design. The optical sensor may be integrated into different configurations within these form factors to optimize physiological monitoring capabilities while maintaining comfort and secure attachment to the chest region.

### RELATED APPLICATIONS

This application claims priority to U.S. Provisional Application No. 63/740,262, filed Dec. 30, 2024, and titled “Optical Sensor Integration into Chest Patch,” which is hereby incorporated by reference in its entirety.

### BACKGROUND

Photoplethysmography (PPG) sensors are widely used for non-invasive monitoring of various physiological parameters. For instance, PPG sensors include optical sensors that measure changes in blood volume in microvascular tissue beds by detecting variations in light absorption. PPG measurements are commonly used for monitoring oxygen saturation, heart rate, respiratory rate, and blood pressure trends as well as other clinical and consumer health applications. PPG sensors may be integrated into wearable devices such as rings, watches, patches, and/or straps.

Using conventional techniques, PPG sensors are typically applied to peripheral sites with high perfusion and thin skin, such as fingers or earlobes. While such sites offer high perfusion and accessibility, the impact of motion artifacts, pressure sensitivity, and ambient light interference at these sites can reduce measurement quality in certain applications, such as monitoring during sleep or physical activity. Alternative PPG sensor placement locations, such as the chest, may be suitable for obtaining PPG measurements, but conventional methods to integrate PPG sensors into mounts for alternative sites are limited. For instance, anatomical and physiological characteristics of different body locations present different challenges for maintaining consistent sensor contact and signal quality. Traditional PPG sensor designs, for instance, are not optimized for the form factors used at alternative mounting sites, which limits effectiveness, computational efficiency, and user comfort.

### BRIEF DESCRIPTION OF THE DRAWINGS

FIG. 1 is a block diagram of a non-limiting example of an environment that is operable to employ techniques for optical sensor integration into a chest patch as described herein.

FIG. 2 depicts a non-limiting example of a monitoring device.

FIG. 3 illustrates various views of a non-limiting example cartridge implementation of the monitoring device.

FIG. 4 illustrates various views of a non-limiting example in-wing configuration of the monitoring device.

FIG. 5 illustrates various views of a non-limiting example snap-in configuration of the monitoring device.

FIGS. 6A and 6B illustrate various views of a non-limiting example auxiliary configuration of the monitoring device.

FIG. 7 depicts a non-limiting example of an integrated optical sensor configuration of the monitoring device.

FIG. 8 depicts an example of different adhesive configurations for integrating an optical sensor into the monitoring device.

FIG. 9 depicts an example of an adhesive configuration for attaching the monitoring device to a person.

FIG. 10 depicts an example of various adhesive configurations for an optical sensor of the monitoring device.

FIG. 11 depicts an example of various lens configurations for use in connection with an optical sensor of the monitoring device.

FIG. 12 depicts an example of a contact structure for the monitoring device.

FIG. 13 depicts an example of an optical sensor configuration for the monitoring device.

FIG. 14 depicts attachment method examples for the monitoring device.

### DETAILED DESCRIPTION

Conventional wearable devices for physiological monitoring often rely on peripheral sites with high perfusion and thin skin, such as fingers or earlobes, for photoplethysmography (PPG) measurements. However, these locations may be unsuitable and/or impractical for various applications, such as continuous monitoring. While modalities that support alternative PPG sensor placement locations, e.g., the chest, are desirable, PPG measurement quality and usability can vary based on sensor placement. By way of example, motion artifacts due to breathing, curved and uneven chest surfaces, and variable tissue composition may present challenges for maintaining PPG sensor contact and/or signal quality. Other physical motions like arm movement, coughing, or posture changes may shift the position of the PPG sensor and disrupt PPG measurements. Furthermore, the chest is susceptible to ambient light exposure, which may introduce noise. Additionally, anatomical structures of the chest region, such as the sternum and ribs, and person-to-person variations in anatomy and physiology, as well as anatomical differences that may exist between genders, may further complicate PPG sensor placement. Based in part on these and other difficulties, traditional sensor designs may not maintain consistent contact and signal quality when applied to alternative body locations, e.g., the chest.

To overcome these limitations, optical sensor integration into a chest patch is described herein. The chest patch may be a wearable device comprising a housing configured to be attached to a chest region of an individual. An adhesive component coupled to the housing secures the housing to the chest region while conforming to contours of the chest region. In some instances, an optical sensor is disposed within the housing. In one or more variations, the optical sensor is positioned within the adhesive component. These designs enable reliable PPG measurements from the chest area, which was previously challenging with conventional approaches.

The wearable device may incorporate additional features to enhance functionality and versatility. By way of example, one or more electrodes may be included to simultaneously detect electrocardiography (ECG), electromyography (EMG), and/or bioimpedance (BioZ) measurements alongside the PPG signal. Various configurations of the housing and adhesive component allow for different integration approaches, such as a cartridge design with radial adhesive regions, a wing-based design with the optical sensor integrated into a wing, a snap-in module that may be removably attached to a rigid mounting interface, and an auxiliary design that separates the optical sensor from the one or more electrodes and/or other components of the wearable device.

To optimize sensor performance, the optical sensor may include a lens with various configurations, such as convex, flat, or concave designs. The wearable device may also incorporate contact components like physical protrusions, springs, foam layers, or inflatable balloons to maintain a desired sensor-skin contact. Multiple attachment methods, including adhesive layers, straps, bands, or suction mechanisms, provide flexibility in how the wearable device is secured to the chest.

This approach offers several advantages over conventional systems. By integrating the optical sensor into a chest patch, the wearable device enables continuous PPG monitoring from a central body location, which may increase measurement stability and consistency relative to conventional techniques. A modular design allows for efficient replacement of adhesive components and/or recharging of electronic components, which extends usability and/or monitoring period of the wearable device. Additionally, the ability to customize the sensor configuration and attachment method to different body types may improve signal quality and user comfort. Accordingly, the wearable device provides a variety of advantages over conventional physiological monitoring technology, such as improved versatility, comfort, and measurement capabilities.

As used herein, the term “continuous” used in connection with measurements, such as PPG measurements, ECG measurements, and the like, may refer to an ability of a device to produce measurements substantially continuously, such that the device may be configured to produce the output measurements at intervals of time (e.g., per hour, per 30 minute interval, per 5 minute interval, per 30 second interval, per second, per half second, and so forth), responsive to an event (e.g., an electrical signal reaching an inflection point such as a peak or a valley), and so forth. The functionality of the device to produce the measurements and/or to record any of a variety of signals may vary without departing from the spirit or scope of the described techniques.

Throughout the figures, elements identified by the same reference numerals may share similar functions, features, and/or designs, but may not be identical unless expressly stated.

In some aspects, the techniques described herein relate to a wearable device for physiological monitoring, including: a housing configured to be attached to a chest region of an individual via an attachment component, the attachment component configured to conform to contours of the chest region; and an optical sensor configured to detect a photoplethysmography (PPG) signal from the chest region of the individual.

In some aspects, the techniques described herein relate to a wearable device, further including at least one of: at least one electrode; an accelerometer; or a temperature sensor.

In some aspects, the techniques described herein relate to a wearable device, wherein the housing includes a cartridge component that includes the optical sensor.

In some aspects, the techniques described herein relate to a wearable device, wherein the attachment component is an adhesive component configured to surround a base of the housing and includes a plurality of petal regions extending radially away from the base.

In some aspects, the techniques described herein relate to a wearable device, wherein the attachment component is an adhesive component including two or more wings, and the optical sensor is integrated into at least one wing of the two or more wings.

In some aspects, the techniques described herein relate to a wearable device, wherein the housing is configured as a snap-in module that is removably attachable to a mounting interface that is coupled to the attachment component.

In some aspects, the techniques described herein relate to a wearable device, further including a main body portion including one or more electrodes configured to detect an electrocardiography (ECG) measurement, and wherein the optical sensor is included in an auxiliary unit that extends from the main body portion.

In some aspects, the techniques described herein relate to a wearable device, wherein the optical sensor includes a lens, the lens having one or more of a convex configuration, a flat configuration, a protruding configuration, or a concave configuration.

In some aspects, the techniques described herein relate to a wearable device, further including a contact component configured to press the optical sensor into the chest region, the contact component including one or more of a spring, a foam layer, a physical protrusion, or an inflatable balloon.

In some aspects, the techniques described herein relate to a wearable device, wherein the attachment component includes an optically clear adhesive configured to bond directly to skin of the individual to provide optical coupling between the optical sensor and the skin.

In some aspects, the techniques described herein relate to a wearable device, wherein the attachment component includes one or more of an adhesive layer, a strap, a band, a textile, a pressure mechanism, a suction mechanism, or a surface energy mechanism.

In some aspects, the techniques described herein relate to a system for physiological monitoring of an individual, including: a wearable monitoring device configured to be attached to a chest region of the individual, including: a substrate having an adhesive disposed thereon configured to secure the wearable monitoring device to the chest region; an optical sensor configured to detect a photoplethysmography (PPG) signal from the chest region of the individual; a transmitter configured to transmit data based on the PPG signal; and a housing configured to house at least the transmitter.

In some aspects, the techniques described herein relate to a system, wherein the housing further houses the optical sensor, and wherein the adhesive includes a plurality of regions extending radially from the housing.

In some aspects, the techniques described herein relate to a system, wherein the adhesive includes two or more wings extending from the housing, and the optical sensor is integrated into at least one wing of the two or more wings.

In some aspects, the techniques described herein relate to a system, wherein the optical sensor is configured as a snap-in module that is removably attachable to a mounting interface coupled to the substrate.

In some aspects, the techniques described herein relate to a system, wherein the wearable monitoring device is divided between a main body portion including the housing and an auxiliary portion including the optical sensor, the auxiliary portion being spaced away from the main body portion, and the system further includes: a flexible connecting element configured to electrically connect the auxiliary portion and the main body portion.

In some aspects, the techniques described herein relate to a system, wherein the optical sensor includes at least one light emitter and at least one photodetector in a protrusion that is configured to press into skin of the individual when the wearable monitoring device is attached to the chest region.

In some aspects, the techniques described herein relate to a wearable monitoring device, including: a substrate configured to be attached to a chest region of an individual via an adhesive component configured to conform to contours of the chest region; one or more electrodes disposed on the substrate and configured to detect electrocardiography (ECG) measurements of the individual; and an optical sensor attached to the substrate and configured to detect a photoplethysmography (PPG) signal from the chest region of the individual.

In some aspects, the techniques described herein relate to a wearable monitoring device, wherein the optical sensor is housed in a snap-in module, and the wearable monitoring device further includes a mounting interface affixed to the substrate, the mounting interface having one or more retention elements shaped to receive the snap-in module.

In some aspects, the techniques described herein relate to a wearable monitoring device, further including: an auxiliary portion spaced away from the substrate; and a flexible connecting element coupled between the auxiliary portion and the substrate, wherein the optical sensor is disposed within the auxiliary portion and attached to the substrate via the flexible connecting element.

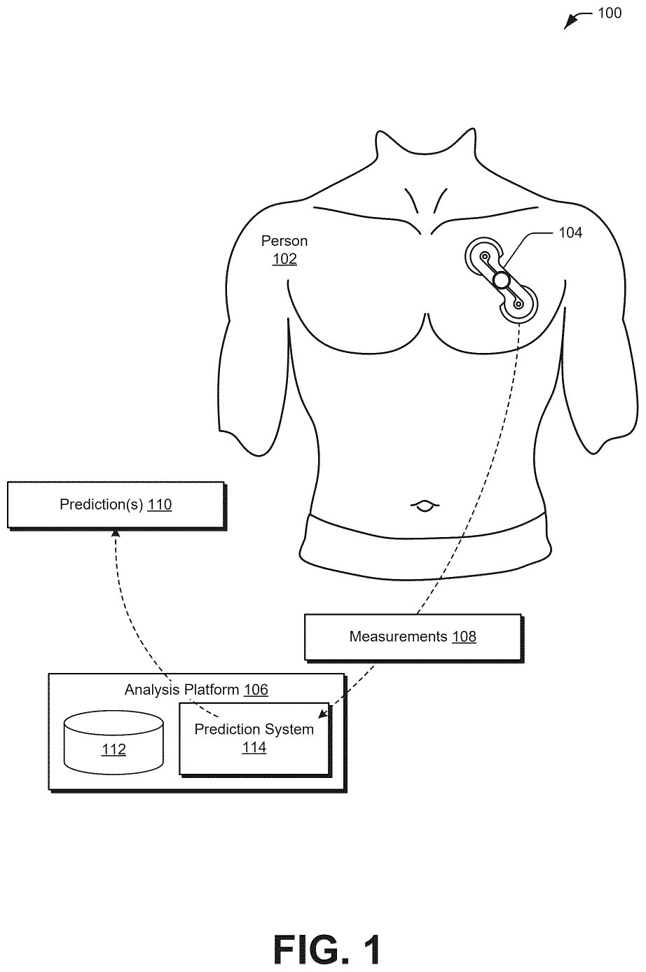

FIG. 1 is a block diagram of a non-limiting example 100 of an environment that is operable to employ techniques for optical sensor integration into a chest patch as described herein. The illustrated example 100 includes a person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102. In some scenarios, for instance, the monitoring device 104 may be provided to record electrical activity from the heart of the person 102 over an observation period, e.g., lasting some number of seconds, minutes, multiple days, and so on. By way of example, a magnitude of electrical potential of the heart of the person 102 may be monitored over time to produce one or more electrocardiograms (ECGs), which may be used to predict any of a variety of events. Alternatively, or in addition, the monitoring device 104 may be used to output measurements 108 (e.g., a time sequence of measurements such as a time sequence of electric potential measurements), which may indicate an observation or be used to generate an assessment, diagnosis, or prediction of one or more events.

In connection with the monitoring device 104, instructions may be provided to the person 102 that instruct the person 102 how to operate the monitoring device 104 and/or how to behave (e.g., sleep, perform activity, etc.) while the wearing monitoring device 104. In one or more implementations, the instructions may be provided as part of a kit, e.g., as written instructions. Alternatively, or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of the observation period has lapsed (e.g., two days) while wearing the monitoring device 104 and/or based on patterns in the aspects of the person 102 being measured.

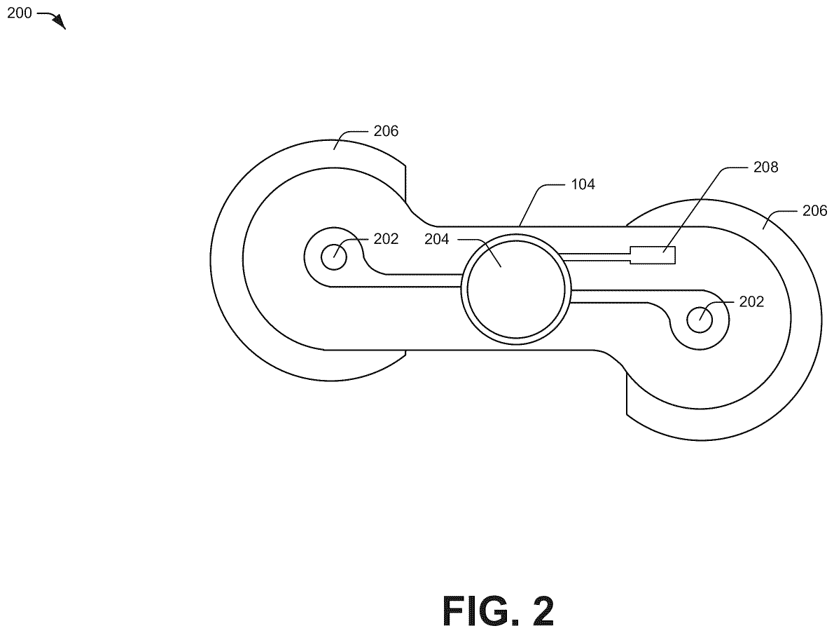

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, a temperature sensor, and a PPG sensor (e.g., to measure and record oxygen saturation (SpO2) and/or produce a photoplethysmogram of the person 102), to name just a few. Certainly, the monitoring device 104 may be configured with any of a variety of types of sensors without departing from the described techniques. The measurements 108 include data received from the one or more sensors.

In the context of the described techniques, the monitoring device includes an optical sensor (e.g., the PPG sensor) for non-invasive monitoring of various physiological parameters, as will be further described herein. By way of example, the optical sensor may be utilized to monitor one or more of heart rate, heart rate variability, blood oxygen saturation, respiration, blood volume, and blood perfusion.

Although the monitoring device 104 may be configured in a similar manner to monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively, or additionally, the monitoring device 104 may have a similar form factor as for clinical settings, but may have different functionality, such as functionality that prevents a wearer from viewing the measurements 108.

In one or more implementations, the monitoring device 104 may be configured to offload the measurements 108 and/or other data during the course of the observation period. By way of example, the monitoring device 104 may offload the measurements 108 by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of sleep apnea.

To the extent that the monitoring device 104 may be configured to store the measurements 108 for an entirety of the observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from a storage of the device.

Accordingly, the monitoring device 104 may be configured with one or more ports to enable wired transmission of the measurements 108 to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternatively or additionally be configured to offload the measurements 108 over one or more wireless connections.

Once the monitoring device 104 produces the measurements 108, the measurements are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from the local storage of the device to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured to generate the one or more predictions 110. For example, the measurements 108 may be processed by a smartphone associated with a user (e.g., the person 102 or another individual associated with the person 102), a smartphone or other dedicated device associated with the monitoring device 104, and/or one or more server computers at a data center or other location that can be utilized by an entity associated with the monitoring device 104, to name just a few.

In one or more implementations, the monitoring device 104 is configured to transmit the measurements 108 to an external device over a wired connection with the external device, e.g., via USB-C or some other physical, communicative coupling. Here, a connector may be plugged into the monitoring device 104, or the monitoring device 104 may be inserted into an apparatus having a receptacle that interfaces with corresponding contacts of the device. The measurements 108 may then be obtained from storage of the monitoring device 104 via this wired connection, e.g., through transfer over the wired connection to the external device. Such a connection may be used in scenarios where the monitoring device 104 is mailed by the person 102 after the observation period, such as to a health care provider, a telemedicine service, a provider of the monitoring device 104, or a medical testing laboratory.

Alternatively, or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with external devices using one or more wireless communication protocols or techniques. By way of example, the monitoring device 104 may communicate with external devices using one or more of Bluetooth® (e.g., Bluetooth® Low Energy links), near-field communication (NFC), Long Term Evolution (LTE™) standards such as 5G, and so forth. The monitoring device 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, or the like. As noted above, examples of such additional measurements include but are not limited to accelerometer data, ECG data, and/or PPG measurements.

In one or more implementations, the analysis platform 106 may be implemented in whole or in part at the monitoring device 104. Alternatively, or in addition, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform includes a storage device 112 and a prediction system 114. In accordance with the described techniques, the storage device 112 is configured to maintain the measurements 108 and/or other measurements or information processed by the prediction system 114 to generate the one or more predictions 110. The storage device 112 may represent one or more databases and/or other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In the illustrated example 100, the prediction system 114 represents functionality to process the measurements 108 to generate the one or more predictions 110. Alternatively, or in addition, the prediction system 114 may output one or more time sequences indicating an observation or prediction of one or more events over time. It is also to be appreciated that, in variations, the prediction system 114 may output different combinations of multiple predictions.

In at least one implementation, the prediction system 114 uses machine learning to generate at least a portion of the one or more predictions 110. In one or more implementations, the one or more predictions 110 may include an assessment or diagnosis related to a health condition or disease state. By way of example and not limitation, the prediction system 114 may include one or more neural networks trained based on the historical measurements and the historical outcome data of a user population. The prediction system 114 may include one or multiple machine learning models (e.g., an ensemble of models). Alternatively, or additionally, the prediction system 114 may include logic (a machine learning model and/or other types of logic) to pre-process the measurements 108, such as to extract various cardiovascular and/or other features from the sequences of measurements. In the illustrated example 100, for instance, the one or more predictions 110 correspond to the output of the prediction system 114.

FIG. 2 depicts a non-limiting example 200 of a monitoring device. The illustrated example 200 depicts the monitoring device 104.

In accordance with the described techniques, the monitoring device 104 includes one or more sensors 202, examples of which include, but are not limited to, one or more pairs of electrodes, an accelerometer, a PPG sensor, temperature sensor(s), and sweat sensors, to name just a few. The monitoring device 104 may also include a transmitter 204, which may be enclosed in a housing, for example. In this example 200, the monitoring device 104 further includes one or more adhesive portions 206 configured as attachment components. In operation, the monitoring device 104 is configured to be applied to the skin via the one or more adhesive portions 206, such that, for example, the one or more sensors 202 are positioned to detect and record the electrical activity of the heart of the person 102, e.g., to produce an electrocardiogram (ECG or EKG).

In at least one implementation, the monitoring device 104 may be removed by peeling the one or more adhesive portions 206 from the skin. It is to be appreciated that the monitoring device 104 may comprise any number of one or more adhesive portions 206. In this example 200, the monitoring device 104 comprises two adhesive portions 206. In one or more implementations, the monitoring device 104 may have three, four, five, or six adhesive portions 206. It is further to be appreciated that the one or more adhesive portions 206 may comprise any shape. In this example 200, the one or more adhesive portions 206 comprise a substantially circular shape. In at least one variation, the one or more adhesive portions 206 may comprise a petal shape, ovular shape, or rectangular shape, just to name a few. Moreover, the one or more adhesive portions 206 may be custom-shaped based on an attachment location (e.g., the chest of the person 102) and/or to increase properties such as wear comfort, sensor function, and attachment strength. It is further to be appreciated that the one or more adhesive portions 206 may comprise a singular, continuous shape (e.g., a substantially rectangular, ovular, winged, or circular shape) that surrounds the transmitter 204.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as the electrical activity of the heart of the person 102. In one or more implementations, the processor further generates one or more communicable packages of data that include one or more of the measurements 108 and/or other measurements, such as accelerometer data and PPG measurements. Alternatively, or additionally, the processor produces and/or causes storage of other data, which may be used for monitoring various physiological states of the person 102.

In implementations where the monitoring device 104 is configured for wireless transmission, the transmitter 204 may transmit the measurements wirelessly as a stream of data to a computing device (e.g., the analysis platform 106). In one or more implementations, for instance, the monitoring device 104 is configured to transfer (e.g., transmit and/or receive) information (e.g., ECG and/or PPG measurements) via a Bluetooth® Low Energy (BLE) connection. Alternatively, or additionally, the monitoring device 104 may buffer the measurements (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

In this example, the monitoring device 104 is depicted as including an optical sensor 208. In some implementations, the optical sensor 208 may include one or more light-emitting components and one or more light-detecting components configured to obtain photoplethysmography (PPG) measurements. For example, the optical sensor 208 may comprise one or more light sources, such as light-emitting diodes (LEDs) and/or laser diodes. The one or more light sources emit light at one or more wavelengths to monitor various physiological parameters. By way of example, the one or more light sources may emit light in the red to infrared spectrum, which penetrates the skin and underlying tissues more efficiently than light having shorter wavelengths (e.g., light within the ultraviolet to orange regions of the spectrum). In at least one variation, however, the one or more light sources of the optical sensor 208 emit light of a shorter wavelength (e.g., green light) in addition to or as an alternative to the longer wavelength light. In one or more implementations, the optical sensor 208 is configured to emit and detect multiple different wavelengths of light to capture different physiological parameters.

The optical sensor 208 may also include one or more photodetectors, such as photodiodes, positioned to detect light reflected from or transmitted through tissue. As the heart pumps blood through the body, the volume of blood in the microvascular bed of the tissue fluctuates. As blood volume in the tissue changes with each heartbeat, the amount of light absorbed or reflected may vary, allowing the optical sensor 208 to detect pulsatile blood flow. By way of example, the photodetector detects these changes in the light absorbed or reflected. The resulting PPG signal may be used to derive various physiological parameters, such as heart rate, blood oxygen saturation, and/or respiratory rate.

The optical sensor 208 may include a lens. In some implementations, the lens may have one or more of a convex configuration, a flat configuration, a domed configuration, or a concave configuration. The specific lens configuration may be selected based on the desired optical properties and performance characteristics of the optical sensor 208.

In one or more implementations, the monitoring device 104 may combine PPG sensing with other modalities, such as ECG or accelerometry, to provide a more comprehensive picture of the physiological state of the person 102. This multi-modal approach may enhance the ability of the monitoring device 104 to detect and monitor various health conditions, including sleep disorders, arrhythmias, or changes in cardiovascular function.

The monitoring device 104 may process raw PPG signals on-board or transmit the data to the analysis platform 106 for further analysis. In some cases, the optical sensor 208 in the monitoring device 104 may be designed for continuous monitoring, allowing for long-term tracking of various health metrics. The monitoring device 104 may also incorporate algorithms to filter out motion artifacts and other noise, improving the accuracy of measurements.

### Optical Sensor Integration into Chest Patch

The figures described below show example configurations for optical sensor integration into a chest patch with relative positioning of the various components and are shown approximately to scale, although other relative dimensions may be used. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

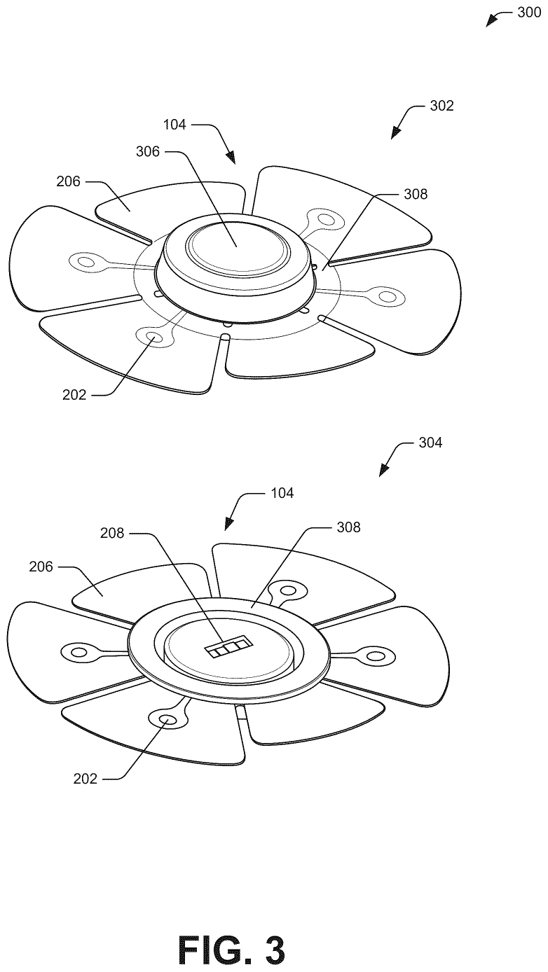

FIG. 3 illustrates various views of a non-limiting example cartridge implementation 300 of the monitoring device 104. The example cartridge implementation 300 is shown in a top view 302 and a bottom view 304.

The example cartridge implementation 300 comprises a housing 306. By way of example, the housing 306 may be configured to contain and/or encapsulate various components of the monitoring device 104 (e.g., the optical sensor 208, the transmitter 204, one or more sensors 202, and/or other electronic components), and the one or more adhesive portions 206 may be used to secure the housing 306 to the person 102. The one or more adhesive portions 206 may comprise a skin-safe acrylic adhesive, a silicone adhesive, a hydrocolloid adhesive, a synthetic rubber adhesive, an optically clear adhesive, or the like. The adhesive used for the one or more adhesive portions 206 (as well as other adhesive components described herein) may be selected by considering wear duration, skin sensitivity, breathability, moisture vapor transmission, placement, and/or activity level, for example.

In the example cartridge implementation 300 shown in FIG. 3, the housing 306 is circular, and the one or more adhesive portions 206 are arranged in a petal-like configuration extending radially from the housing 306. However, it is to be appreciated that the example cartridge implementation 300 may include any number of the one or more adhesive portions 206, and the housing 306 and/or the one or more adhesive portions 206 may comprise other shapes. By way of example, the one or more adhesive portions 206 may be designed to conform to contours of the chest region, which may provide a secure and comfortable fit for the monitoring device 104. In at least one implementation, the one or more adhesive portions 206 may comprise the one or more sensors 202, such as electrodes configured to detect electrocardiography (ECG) measurements. An additional benefit of having electrodes in one or more adhesive portions 206 is the ability to place electrodes in any number of vectors as appropriate for a given application. For example, two electrodes on opposite one or more adhesive portions 206 may measure ECG, and one or more other electrodes may measure bioimpedance (BioZ) or impedance cardiography (ICG). As another example, electrodes on four different adhesive portions 206 may be provided to measure multi-lead ECG. Moreover, it is to be appreciated that the one or more adhesive portions 206 may comprise a substrate having an adhesive material affixed thereon, e.g., as a coating. In the example cartridge implementation 300, the housing 306 contains at least the optical sensor 208.

In the top view 302, the housing 306 is shown positioned at a center of the example cartridge implementation 300. A housing base 308 is positioned adjacent to the housing 306 and provides a transition between the housing 306 and the one or more adhesive portions 206. In some cases, the housing base 308 may be configured to envelop the housing 306. The housing 306 may attach to the housing base 308 and/or the one or more adhesive portions 206 by an adhesive and/or by a mechanical fastening method, e.g., interlocking tabs, screws, clips, and the like.

In the bottom view 304, the optical sensor 208 is shown integrated into the housing 306. The optical sensor 208 may be positioned to maintain contact with the skin of the person 102 when the monitoring device 104 is worn, which allows the optical sensor 208 to detect a photoplethysmography (PPG) signal from a chest region of the person 102.

The housing 306 may have a shape and a size that is commensurate with the shape and size of the various components that the housing 306 is configured to contain. Alternatively, the housing 306 may have a shape and a size that is larger than the shape and size of the various components that the housing 306 is configured to contain. In some implementations, the housing 306 is rigid or semi-rigid and protects the various components that the housing 306 is configured to contain. The housing 306 may be waterproof and/or dustproof. In the example cartridge implementation 300, the housing 306 is configured as a cap that covers at least top portions and side portions of the various components, such as the optical sensor 208.

The example cartridge implementation 300 may allow for modular construction of the monitoring device 104. A modular construction of the monitoring device 104 may provide for flexibility and customization as components may be swapped depending on the measurement needed, e.g., ECG, accelerometry, PPG, and/or temperature. Additionally, a modular construction of the monitoring device 104 may provide for incremental updates and/or replacement of components individually without replacing the monitoring device 104 in its entirety. In some implementations, a force-providing element, such as a spring or foam element may be positioned to apply force to the housing 306. This force may help maintain consistent contact between the optical sensor 208 and the skin of the person 102, which may improve a quality of the PPG signal detection. The force may also permit the optical sensor 208 to conform to curves and irregularities of anatomical structures along the chest of the person 102. The spring or foam element may also act to buffer movements of or impacts across the chest of the person 102. In other implementations, the spring or foam element may be positioned within the housing 306, such as to apply force directly to the optical sensor 208 or another component contained by the housing 306.

The configuration of the example cartridge implementation 300 enables the monitoring device 104 to function as a wearable device for physiological monitoring. By integrating the optical sensor 208 within the housing 306 and providing a secure attachment mechanism through the one or more adhesive portions 206, the example cartridge implementation 300 facilitates reliable PPG signal detection from the chest region of the person 102.

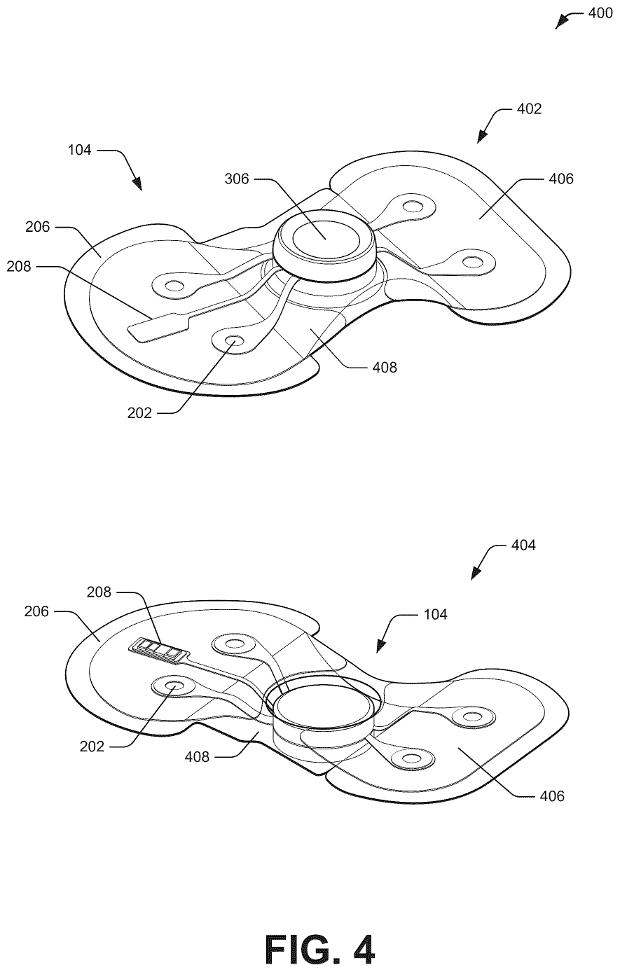

FIG. 4 illustrates various views of an example in-wing configuration 400 of the monitoring device 104. The example in-wing configuration 400 is shown in a top view 402 and a bottom view 404 demonstrating how the optical sensor 208 is integrated into a wing-shaped configuration.

In the in-wing configuration 400, the optical sensor 208 is integrated into a wing-shaped structure with two wing elements 406. The example in-wing configuration 400, for instance, includes the two wing elements 406 extending laterally from the housing 306. The two wing elements 406 may be configured to conform to a surface of the person 102. In the present example, each of the two wing elements 406 has a rounded, elongated shape that tapers outward as it extends away from the housing 306, although other shapes may be used. In some cases, the two wing elements 406 may further include the one or more sensors 202. It is to be appreciated that although one optical sensor 208 is shown, in at least one variation, the in-wing configuration 400 includes more than one optical sensor 208, such as two optical sensors 208 integrated into the same wing or into different wings of the two wing elements 406.

As with the example cartridge implementation 300 of FIG. 3, the housing 306 of the example in-wing configuration 400 may be configured to contain various components of the monitoring device 104, such as the transmitter 204, the one or more sensors 202, and/or other electronic components (e.g., a printed circuit board). In the example in-wing configuration 400, the housing 306 contains at least the transmitter 204. A housing bridge 408 connects the housing 306 to each of the two wing elements 406, which include the one or more adhesive portions 206. The housing bridge 408 may be flexible to allow the two wing elements 406 to move independently of the housing 306. In this configuration, the housing 306 may be elevated with respect to the two wing elements 406, as shown in the top view 402, and may float above the skin of the person 102 while the two wing elements 406 conform to the chest region. This decoupling may reduce motion artifacts and improve signal quality by limiting the transmission of external energy to the sensor-skin interface.

In the example in-wing configuration 400, the optical sensor 208 is integrated into a particular wing of the two wing elements 406, thereby taking advantage of an existing adhesive footprint. In some implementations, the example in-wing configuration 400 may integrate optically clear adhesives, which may cover the outside surface of the optical sensor. The bottom view 404 provides a view of an underside of the example in-wing configuration 400, demonstrating how the optical sensor 208 is positioned to make contact with skin of the person 102 when the monitoring device 104 is worn. The bottom view 404 further shows how the one or more sensors 202 may be positioned in the two wing elements 406 to make contact with the skin of the person 102. In some implementations, the one or more sensors 202 may include electrodes configured to detect ECG and/or other electrical measurements.

The example in-wing configuration 400 demonstrates an approach for incorporating the optical sensor 208 into a flexible, body-conforming structure that may be secured to the person 102 while maintaining desired sensor contact. This configuration allows the monitoring device 104 to adapt to body contours while housing various electronic components for obtaining PPG and/or ECG measurements.

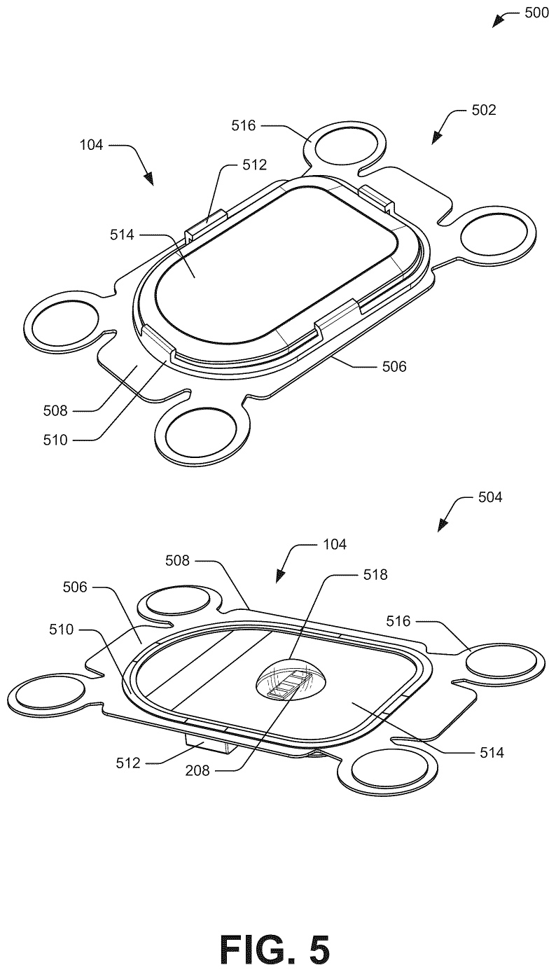

FIG. 5 illustrates various views of a non-limiting example snap-in configuration 500 of the monitoring device 104. The example snap-in configuration 500 is shown in a top view 502 and a bottom view 504.

The example snap-in configuration 500 comprises an adhesive surface 506 (e.g., an attachment component for attaching the monitoring device 104 to the person 102) on a substrate 508 that is coupled to a mounting interface 510 having one or more retention elements 512. The mounting interface 510 is shaped to receive a snap-in module 514 that houses the optical sensor 208. By way of example, although the top view 502 and the bottom view 504 both show the snap-in module 514 positioned within the mounting interface 510, the snap-in module 514 is configured to be removable from the mounting interface 510. The mounting interface 510 may have a shape that complements a shape of the snap-in module 514. By way of example, the mounting interface 510 may have a circular, oval, rectangular, square, or capsule-shaped profile that corresponds to a profile of the snap-in module 514. The mounting interface 510 defines an aperture through which the optical sensor 208 may contact skin of the person 102 when the snap-in module 514 is received by the mounting interface 510. The mounting interface 510 may be rigid or semi-rigid, for example.

In at least one implementation, the one or more retention elements 512 are configured to engage with the snap-in module 514 to removably secure the snap-in module 514 within the mounting interface 510. The one or more retention elements 512 may include clips, latches, detents, friction-fit elements, and/or the like. Although four retention elements are shown in the example snap-in configuration 500, it is to be appreciated that the mounting interface 510 may include any number of retention elements 512.

It is to be appreciated that in at least one variation, the snap-in module 514 is designed to stay retained by the mounting interface 510 irreversibly. In this case, an entirety of the snap-in configuration 500, including the substrate 508 and the mounting interface 510, may be removed and replaced. It may also be possible to remove the snap-in module and, in doing so, break the retention elements 512. In such examples, the mounting interface 510 may be replaced, such as by replacing the substrate 508 in combination with the mounting interface 510 or replacing the mounting interface 510 while the substrate 508 remains positioned on the person 102.

The substrate 508 may further include multiple extending regions 516 arranged to secure the monitoring device 104 to a chest region of the person 102. The substrate 508 may include any number of extending regions 516. For instance, the example snap-in configuration 500 illustrates how the substrate 508 may include four extending regions 516 positioned around a perimeter of the substrate 508. Additionally, the multiple extending regions 516 may comprise any shape. For instance, the example snap-in configuration 500 illustrates how the multiple extending regions 516 may comprise a generally circular shape. The extending regions 516 can be utilized for electrodes configured to detect ECG or other electrical measurement, or to provide additional adhesive to securely adhere the device to the person 102. An additional benefit of having electrodes in the extending regions 516 is the ability to place electrodes in any number of vectors as appropriate for a given application. For example, two electrodes on opposite extending regions 516 may measure ECG while the two others measure BioZ. As another example, electrodes on four different corners may be provided to measure multi-lead ECG.

The bottom view 504 shows the optical sensor 208 positioned in the central region of the monitoring device 104 via the snap-in module 514 and the mounting interface 510. In variations, the optical sensor 208 may be positioned anywhere along the skin-facing side of the monitoring device 104. The example snap-in configuration 500 incorporates a modular configuration where components (e.g., the optical sensor 208) may be removably attached via the snap-in module 514. By way of example, the snap-in module 514 may be configured to house various components of the monitoring device 104, such as the optical sensor 208, the transmitter 204, a battery, and/or other electronic components.

The bottom view 504 also shows the optical sensor 208 positioned under a lens 518. The lens 518 may improve PPG signal quality by focusing the emitted light into the skin, increasing penetration depth, and/or reducing scatter. The lens 518 may also provide greater sensitivity by concentrating the reflected light received by a photodetector of the optical sensor 208, thereby increasing the signal-to-noise ratio. Furthermore, the lens 518 may also increase energy efficiency of the monitoring device 104 by directing light into the skin more effectively, thereby reducing the power utilized by the light source. The lens 518 may enable miniaturization of the optical sensor 208 and/or the monitoring device 104 by optimizing light paths, for instance. The lens 518 is shown with a convex shape, but may comprise other interface shapes like concave, flat/flush, or another shape.

The overall form factor of the example snap-in configuration 500 may be compact, reusable, and designed to conform to body contours while maintaining secure attachment through the multiple extending regions 516. By way of example, the snap-in module 514 may be replaced while the substrate 508 is affixed to the person 102, or the same snap-in module 514 may be used with multiple instances of the substrate 508 that are worn at different times and/or by different people. By way of example, the person 102 may remove the snap-in module 514 to recharge its battery without removing the substrate 508 from the skin. As another example, the snap-in module 514 may be cleaned and/or reset from user to user, as the snap-in module 514 may be placed on a new user with a new substrate 508. As yet another example, a user (e.g., the person 102) removes the snap-in module 514 from the mounting interface 510 of a worn substrate 508. The user then applies a new substrate 508 with a fresh adhesive surface 506 to the chest region of the person 102 and reinserts the snap-in module 514 into the mounting interface 510 of the new substrate 508, effectively replacing the adhesive surface 506 while retaining the electronic components of the monitoring device 104, including the optical sensor 208. This approach may provide advantages such as extended wear time, reduced waste, and improved cost-effectiveness by allowing users to replace the substrate 508 (e.g., in response to the adhesive surface 506 becoming compromised) while reusing the electronic components of the monitoring device 104. The snap-in module 514 may thus include a self-contained, encapsulated module for the optical sensor 208 that supports reuse across multiple substrates 508.

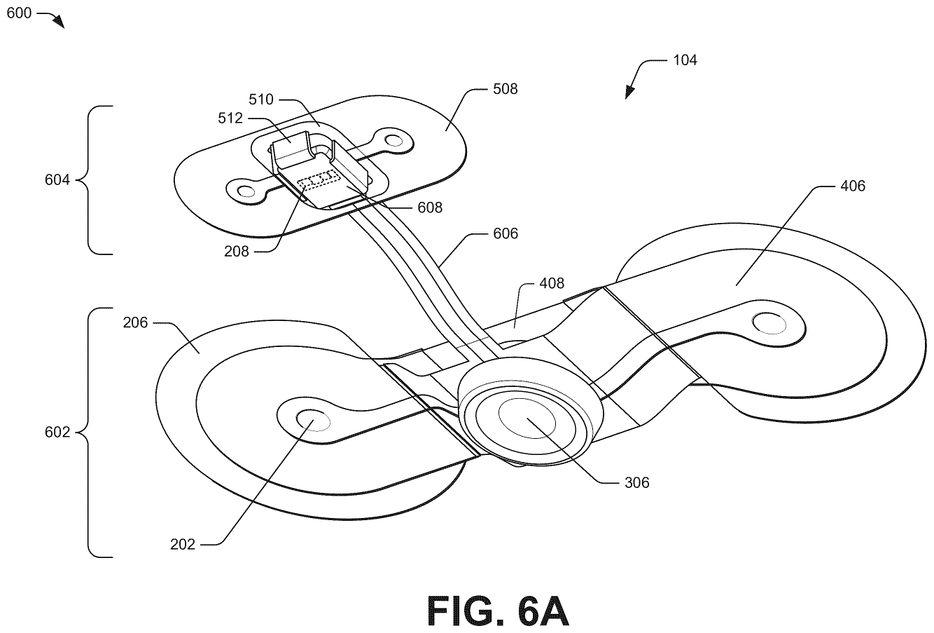

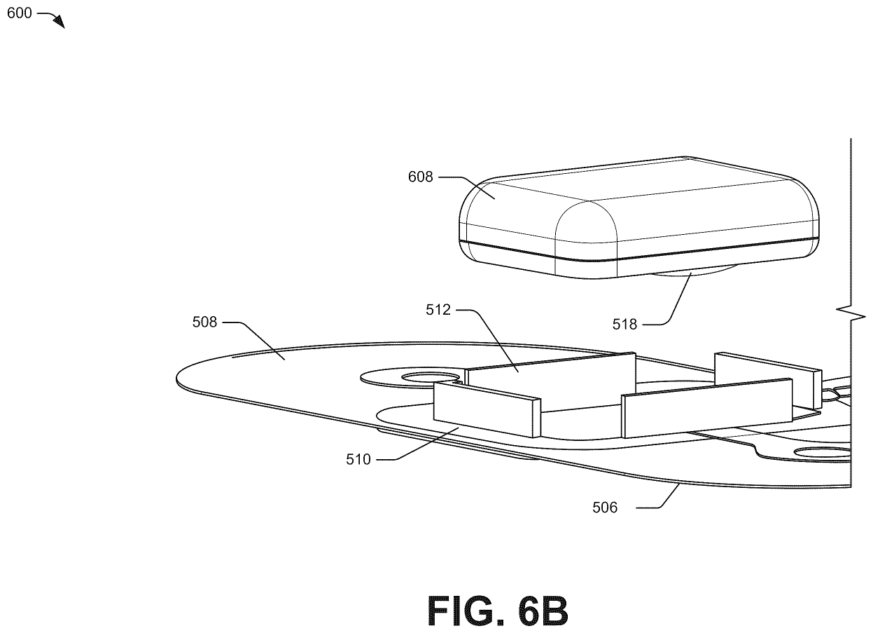

FIGS. 6A and 6B illustrate various views of a non-limiting example auxiliary configuration 600 of the monitoring device 104. FIG. 6A shows an isometric view of the auxiliary configuration 600, and FIG. 6B shows a side perspective view of the auxiliary configuration 600.

The auxiliary configuration 600 comprises the monitoring device 104 divided between a main body portion 602 and an auxiliary portion 604 connected by a flexible connecting element 606. The flexible connecting element 606 may electronically, communicatively, and/or physically connect (e.g., couple) the main body portion 602 and the auxiliary portion 604. By way of example, the flexible connecting element 606 may include wires that provide an electrical connection between the main body portion 602 and the auxiliary portion 604. Alternatively, or in addition, the flexible connecting element 606 may carry an electrical signal or enable electronic communication between the main body portion 602 and the auxiliary portion 604, such as between the auxiliary portion 604 and the transmitter 204. Additionally, the flexible connecting element 606 may physically tether the auxiliary portion 604 to the main body portion 602 such that the auxiliary portion 604 is spaced apart from the main body portion 602 at a distance that is no greater than a length of the flexible connecting element 606. The flexible connecting element 606 may be adhered to the person 102, may be floating, or may have a mix of adhered or floating regions along the length of the flexible connecting element 606.

In the present example, the main body portion 602 includes the housing 306 positioned at a central location, and the two wing elements 406 extend laterally from the housing 306. The two wing elements 406 are connected to the housing 306 via the housing bridge 408, e.g., as described above with respect to the in-wing configuration 400 of FIG. 4, and may include the one or more adhesive portions 206 configured to secure the main body portion 602 to the chest region of the person 102 and the one or more sensors 202.

The auxiliary portion 604 is spaced away from the main body portion 602 and includes an auxiliary sensing unit 608. The auxiliary sensing unit 608 includes the optical sensor 208 in this example, shown as dashed outlines in FIG. 6A due to its location within the auxiliary sensing unit 608. In some cases, the auxiliary sensing unit 608 may include additional sensors of the one or more sensors 202, such as additional electrodes configured for ECG or other electrical measurement. These electrodes may enable measurement of different ECG vectors or other electrical measurements such as bioimpedance.

In the present example, the auxiliary sensing unit 608 includes a removable configuration similar to the snap-in module 514 described above with respect to FIG. 5. By way of example, the auxiliary portion 604 includes the substrate 508 and the adhesive surface 506 on the bottom/skin-facing surface (see FIG. 6B) to adhere the auxiliary portion 604 to the person 102, and the auxiliary sensing unit 608 is mounted to the substrate 508 via the mounting interface 510 having the one or more retention elements 512. Removal of the auxiliary sensing unit 608 from the mounting interface 510 is shown in FIG. 6B. In at least one variation, however, the auxiliary sensing unit 608 is directly affixed to the substrate 508, e.g., via an adhesive, a weld, a mechanical fastener, integrated between adhesive/film layers, and/or the like. In some implementations, the flexible connecting element 606 may include a detachable connector, allowing the auxiliary sensing unit 608 to be unplugged from the main body portion 602 and replaced.

As shown in FIG. 6B, the auxiliary sensing unit 608 may include the lens 518 over the optical sensor 208. In various implementations, the auxiliary sensing unit 608 contains additional components beyond the optical sensor 208. For example, an analog front end (AFE) may be included in the auxiliary sensing unit 608 along with the optical sensor 208. Including the AFE in the auxiliary sensing unit 608 may minimize the distance over which analog signals travel, which may improve signal quality.

The auxiliary configuration 600 demonstrates an approach for incorporating the optical sensor 208 into a separate, extended unit while maintaining connection to the housing 306 of the monitoring device 104. This configuration provides flexibility in placement of the optical sensor 208 with respect to the main body portion 602 and the one or more sensors 202 included therein, which may improve a quality of both optical and electrical measurements obtained by the monitoring device 104.

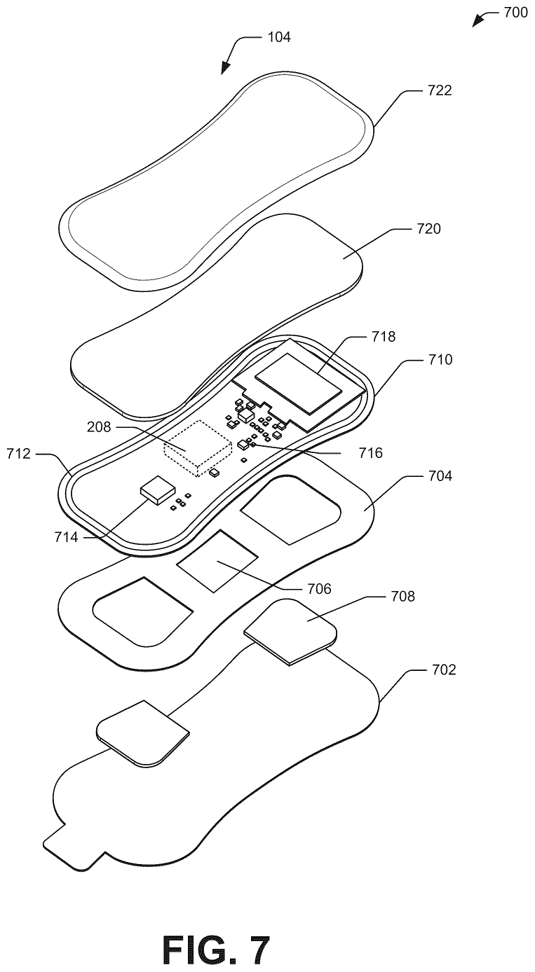

FIG. 7 depicts an example integrated optical sensor configuration 700 of the monitoring device 104. In this example, the example integrated optical sensor configuration 700 is depicted as an “all-in-one” flexible configuration of the monitoring device 104 and is shown as an exploded view. The example integrated optical sensor configuration 700 includes a removable liner 702 configured to protect an adhesive layer 704 prior to placement on the skin of the person 102. The adhesive layer 704 may be configured to affix the example integrated optical sensor configuration 700 to the person 102, for example, when the removable liner 702 is removed. The adhesive layer 704 optionally includes a region 706 of optically clear adhesive configured to cover the optical sensor 208 and ensure strong adhesive coupling between the optical sensor 208 and the skin of the person 102. When the region 706 is omitted, the optical sensor 208 may contact the skin of the person 102 directly.

The example integrated optical sensor configuration 700 further includes a conductive hydrogel layer 708 (e.g., for obtaining ECG measurements) positioned in cutout portions of the adhesive layer 704 and configured to align with electrodes (not shown in FIG. 7) positioned on an underside of a substrate layer 710. The substrate layer 710 may comprise a flexible substrate material configured for printed circuit boards, such as polyimide. Alternatively, the substrate layer 710 may comprise a rigid material, such as fiberglass and epoxy resin laminate (e.g., FR4), or a semi-rigid material. The optical sensor 208 is shown mounted on the underside of the substrate layer 710 (as represented by dashed lines in the integrated optical sensor configuration 700 of FIG. 7). Additional electronic components are mounted on the substrate layer 710, including a flexible antenna 712, an analog front end 714, a wireless communication component 716, and a battery 718. A spacer 720 is optionally disposed above the substrate layer 710. By way of example, the spacer 720 (e.g., a foam spacer) may shield and/or protect the electronic components disposed on the substrate layer 710. A thin-film layer 722 is disposed above the spacer 720. The thin-film layer 722 may be polyurethane, polyethylene terephthalate, or another type of plastic or thermoplastic polymer resin that covers and/or seals the integrated optical sensor configuration 700. The example integrated optical sensor configuration 700 may provide self-contained electronics that simplify waterproofing and mechanical and electrical integration of the monitoring device 104, for example. In at least one implementation, the removable liner 702 may include cutouts or an optically clear material underneath the optical sensor 208.

The following examples and corresponding figures provide a variety of examples of additional properties, features, configurations, and/or implementations of the techniques described herein. The examples below are provided by way of example and not limitation and it should be understood that many variations are possible based on the disclosure herein.

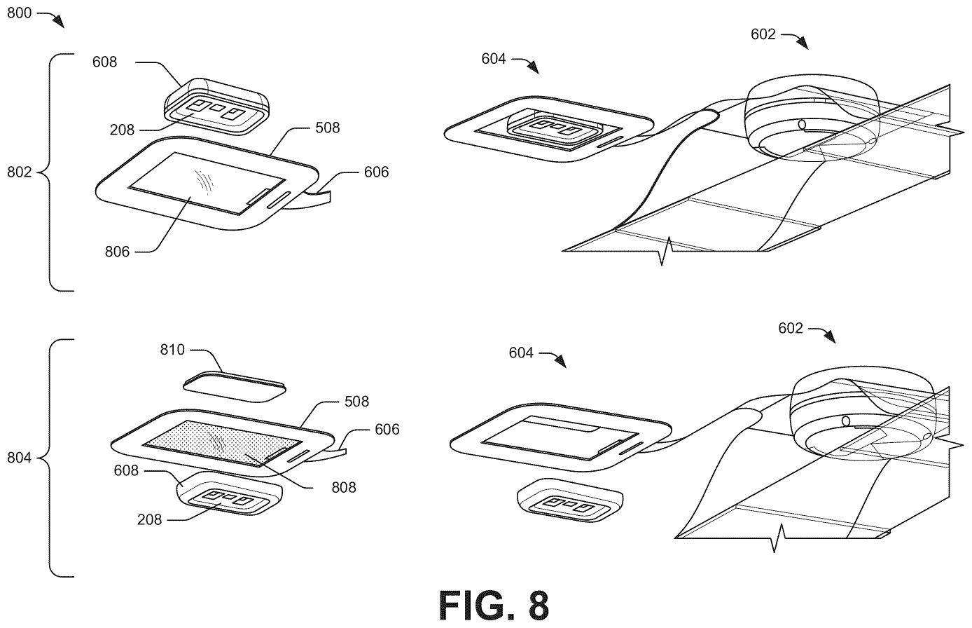

FIG. 8 depicts an example 800 of different adhesive configurations for integrating the optical sensor 208 into the monitoring device 104. The adhesive configurations shown in the example 800 may be used in connection with any of the configurations described herein. The example 800 will be described with respect to the example auxiliary configuration 600, but this is by way of illustration and not limitation.

The example 800 includes an integrated configuration 802 and a sandwich configuration 804. In the integrated configuration 802, the auxiliary sensing unit 608 is positioned above the substrate 508 and an optically clear adhesive 806 disposed within the substrate 508. This enables the optically clear adhesive 806 to bond directly to the optical sensor 208 of the auxiliary sensing unit 608, eliminating air gaps between the optical sensor 208 (or a lens 518 covering the optical sensor 208, not shown in FIG. 8) and skin. By way of example, the optically clear adhesive 806 may affix the auxiliary sensing unit 608 to the substrate 508 and/or to the person 102. The optically clear adhesive 806 may be a double-sided adhesive, for example. Alternatively, the auxiliary sensing unit 608 may be affixed to the substrate 508 via another means (e.g., the mounting interface 510), and the optically clear adhesive 806 may affix the optical sensor 208 to the body. In yet another variation, a lens is included in addition to or as an alternative to the optically clear adhesive 806. The integrated configuration 802 may provide consistent optical coupling and minimize light leakage while enabling through-housing electrical connections (e.g., via the flexible connecting element 606) via, for example, spring pins.

In the sandwich configuration 804, at least a portion of the auxiliary sensing unit 608 that includes the optical sensor 208 is positioned below the substrate 508. The substrate 508 further includes an overlay adhesive 808, which may or may not be optically clear. By way of example, because the optical sensor 208 is positioned below the substrate 508 and the overlay adhesive 808, light transmitted and received by the optical sensor 208 will not pass through the overlay adhesive 808. In this example, a cap portion 810 of the auxiliary sensing unit 608 covers the auxiliary sensing unit 608 from above, “sandwiching” the overlay adhesive 808. The flexible connecting element 606 extends from the auxiliary sensing unit 608 to provide electrical connection between the auxiliary sensing unit 608 and the main body portion 602. This configuration may provide a thin opto-mechanical sensor integration under the overlay adhesive 808 and leverage internal electrical connections between the auxiliary portion 604 and the main body portion 602.

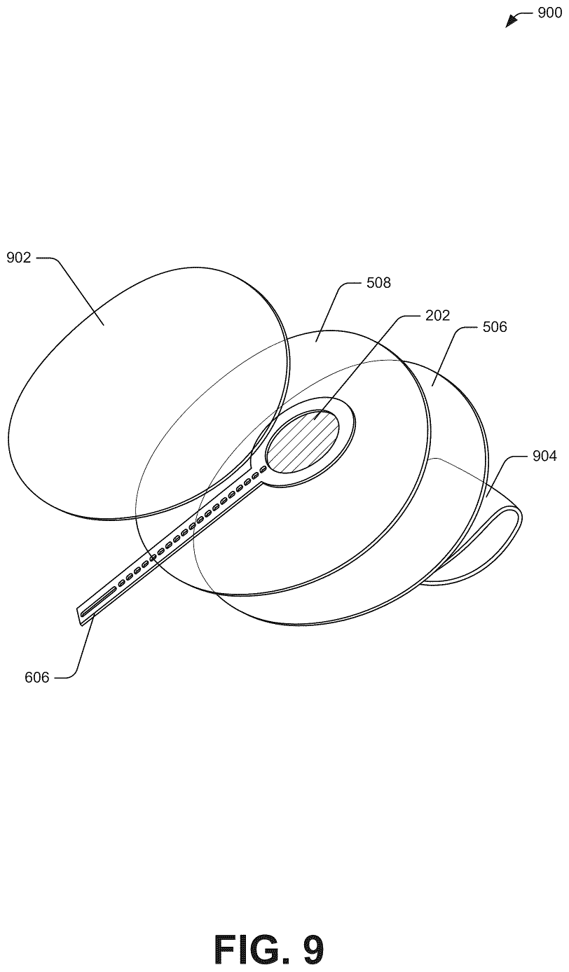

FIG. 9 depicts an example of an adhesive configuration 900 for attaching any, multiple, or all components of the monitoring device 104 to the person 102. The adhesive configuration 900 is shown in an exploded view in FIG. 9 and may be used in connection with any of the configurations described herein. By way of example, the adhesive configuration 900 will be described with respect to the example auxiliary configuration 600. The adhesive configuration 900 includes the substrate 508 having the adhesive surface 506 disposed thereon, e.g., on an underside of the substrate 508 that is configured to be in contact with the skin of the person 102. The flexible connecting element 606 extends from the substrate 508 to provide electrical connection between sensing components (shown generally in FIG. 9 as the one or more sensors 202) and the main body portion 602 of the monitoring device 104 (not shown in FIG. 9). The adhesive configuration 900 further includes a protective cover 902 configured to overlay the substrate 508. The protective cover 902 may include medical tape, elastic polyurethane, silicone film, polyethylene film, a fabric overlay, a breathable membrane, and/or the like, and may protect the substrate 508 and components disposed therein from exposure and/or degradation. The protective cover 902 may extend a wear time of the monitoring device 104, for example. A release liner 904 is disposed beneath the adhesive surface 506 and is configured to be removed to expose the adhesive surface 506 prior to attachment to the person 102.

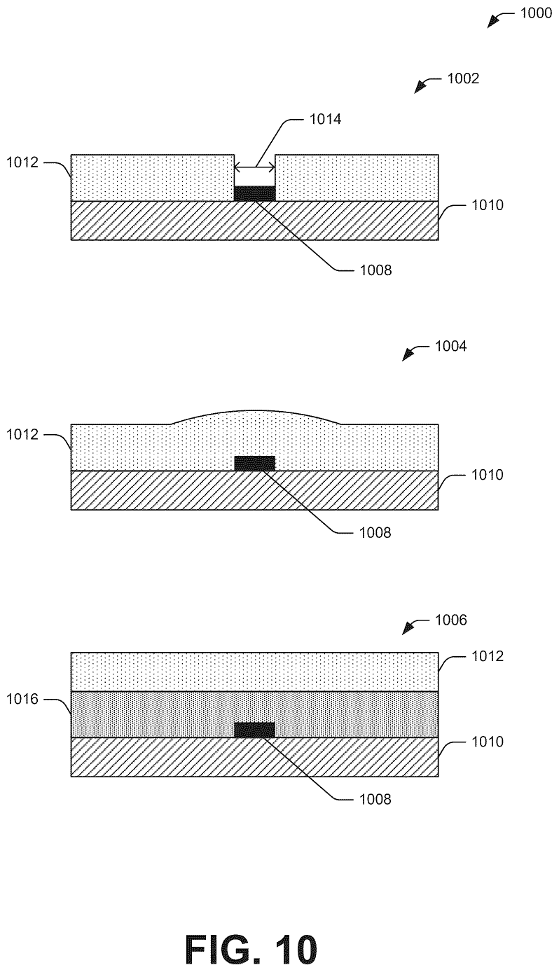

FIG. 10 depicts an example 1000 of various adhesive configurations for an optical sensor of the monitoring device. The example 1000 includes a first adhesive configuration 1002, a second adhesive configuration 1004, and a third adhesive configuration 1006. In each configuration, an emitter/photodiode 1008 is shown disposed on a substrate 1010 (e.g., an opaque substrate), with a skin-facing surface toward a top of each diagram. The emitter/photodiode 1008 represents an emitter configured to emit light (e.g., an LED, laser diode, or other) or a photodiode (or other photodetector) configured to detect light, as the emitter and photodiode may be arranged similarly. Each configuration further includes an optically clear adhesive 1012 configured to attach to the skin of the person 102.

In the first adhesive configuration 1002, the optically clear adhesive 1012 is disposed adjacent to the emitter/photodiode 1008. An opening 1014 is formed in the optically clear adhesive 1012 above the emitter/photodiode 1008. The opening 1014 enables light emitted or received by the emitter/photodiode 1008 to be transmitted without passing through the optically clear adhesive 1012. In addition, the opening 1014 may provide an area for skin or sweat to occupy, which may create a good coupling surface between the emitter/photodiode 1008 and skin.

In the second adhesive configuration 1004, the optically clear adhesive 1012 covers the emitter/photodiode 1008. The second adhesive configuration 1004 differs from the first adhesive configuration 1002 in that the optically clear adhesive 1012 extends continuously over the emitter/photodiode 1008, without the opening 1014. Accordingly, light emitted or received by the emitter/photodiode 1008 will be transmitted through the optically clear adhesive 1012, and the emitter/photodiode 1008 is encapsulated. In at least some implementations, an air gap may exist between the emitter/photodiode 1008 and the optically clear adhesive 1012, such as above and/or on either side of the emitter/photodiode 1008. However, it may be advantageous to reduce or eliminate the air gap to improve optical coupling.

In the third adhesive configuration 1006, the emitter/photodiode 1008 is encased by an optically clear resin 1016 between the substrate 1010 and the optically clear adhesive 1012. The optically clear resin 1016 provides direct optical coupling between the emitter/photodiode 1008 and the optically clear adhesive 1012, which may provide consistent optical coupling, e.g., by ensuring there are no air gaps. Accordingly, light emitted by the emitter/photodiode 1008 will pass through the optically clear resin 1016 and the optically clear adhesive 1012. In one or more implementations, the optically clear adhesive 1012 and the optically clear resin 1016 may minimize light leakage and reduce application pressure variability when included in the optical sensor 208.

In at least one variation, the third adhesive configuration 1006 may not include the optically clear adhesive 1012, or the optically clear adhesive 1012 may include the opening 1014 directly above the emitter/photodiode 1008, similar to the first adhesive configuration 1002. In one or more other variations, the third adhesive configuration 1006 may include a glass layer directly above the emitter/photodiode 1008. When the optically clear adhesive 1012 is omitted, adhesive may be adjacent to or surrounding the third adhesive configuration 1006 for attachment to the skin of the person 102.

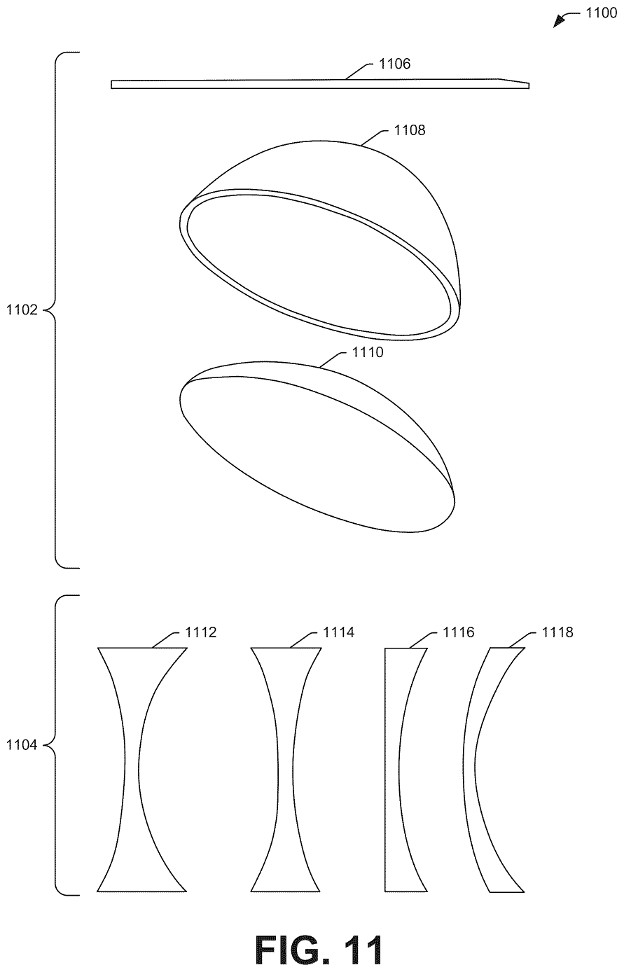

FIG. 11 depicts an example 1100 of various lens configurations for use in connection with the optical sensor 208 of the monitoring device 104. The example 1100 includes lens geometries 1102 and lens profiles 1104. The lens geometries 1102 and the lens profiles 1104 may have a variety of shapes and material compositions, e.g., acrylic, polycarbonate, glass, sapphire, and the like that may be used for the lens 518.

The lens geometries 1102 include a flat lens 1106, a first domed lens 1108, and a second domed lens 1110. The flat lens 1106 has a planar configuration that may provide for a simpler design that is easier to manufacture and integrate into the optical sensor 208. The flat lens 1106 may also provide uniform contact with a skin surface, which may minimize air gaps. The first domed lens 1108 and the second domed lens 1110 have convex configurations that may improve skin conformity across a wider area, thereby reducing stray light leakage. A convex configuration, for instance, may capture more diffuse reflected light or help direct light into and out of the skin, thereby increasing signal strength. A convex configuration may be well-suited for variable skin surfaces having contours, where contact with the flat lens 1106 may be inconsistent. A convex configuration may also enhance a signal-to-noise ratio by reducing ambient light intrusion. The first domed lens 1108 and the second domed lens 1110 can be half-ball lenses or lenses of greater or lesser height. The lens geometries 1102 may include additional optically clear resin to eliminate air gaps between the optical components and the lens.

By way of example, the first domed lens 1108 or the second domed lens 1110 may protrude from the monitoring device 104 (or a portion thereof) toward the person 102, such as demonstrated in the auxiliary configuration 600 of FIG. 6B and the snap-in configuration 500 of FIG. 5. Protruding configurations may be utilized to create tight optical coupling at a specific point on the skin, thereby reducing air gaps. A localized indentation made by the protruding configuration may further help stabilize contact pressure, thereby improving consistency across readings. The localized indentation may reduce motion artifacts by anchoring the optical sensor 208 locally. The localized indentation may also direct light into a defined tissue volume, thereby enhancing repeatability. A curvature radius of the lens 518 may be selected to balance qualities like penetration depth and signal-to-noise ratio against other qualities like design complexity and contact stability, for instance.

The lens profiles 1104 include a concave lens 1112, a biconcave lens 1114, a plano-concave lens 1116, and a negative meniscus lens 1118. The concave lens 1112 has inwardly curving surfaces. The biconcave lens 1114 has a symmetrical configuration with both surfaces curving inward by a same amount. The plano-concave lens 1116 has one flat surface and one concave surface. The negative meniscus lens 1118 has one convex surface and one concave surface, with the concave surface having a greater curvature than the convex surface. This creates a lens that is thinner in the middle than at the edges. The negative meniscus lens 1118 may spread light rather than focusing it, while the meniscus shape (e.g., one convex surface, one concave surface) may help reduce optical aberrations compared to the concave lens 1112, the biconcave lens 1114, or the plano-concave lens 1116. Alternatively, the negative meniscus lens 1118 may have a convex surface having a greater curvature than the concave surface, creating a lens that is thinner at the edges than in the middle. The lens profiles 1104 may be selected based on desired light spreading or focusing properties, for example, to help manage the angle at which light enters/exits tissue.

Accordingly, the various lens configurations shown in the example 1100 may be selected based on desired optical properties and performance characteristics for the monitoring device 104.

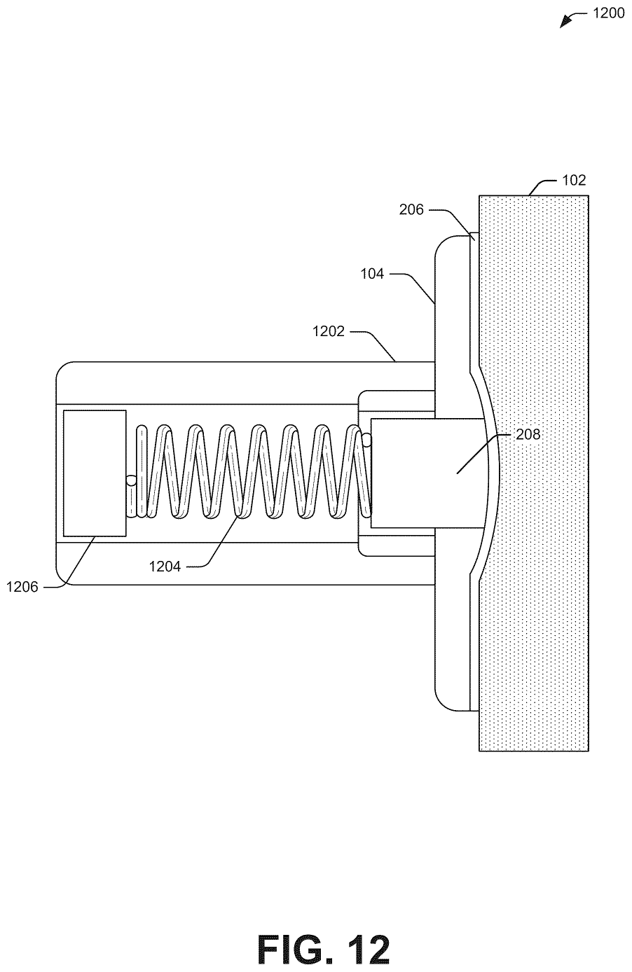

FIG. 12 depicts an example 1200 of a contact structure 1202 for the monitoring device 104. The example 1200 includes the monitoring device 104 affixed to the person 102 via the one or more adhesive portions 206. The contact structure 1202 is positioned within the monitoring device 104 and, in this example, includes a spring 1204 configured to apply a force toward the skin of the person 102. A spring stop 1206 is positioned at one end of the spring 1204 and provides a mounting point for the spring 1204 within the contact structure 1202.

The optical sensor 208 is positioned between the spring 1204 and the skin of the person 102. The spring 1204 applies pressure to the optical sensor 208, pressing the optical sensor 208 against the skin of the person 102. This configuration may help ensure consistent contact between the optical sensor 208 and the skin, which may improve a quality of PPG signal detection. The spring 1204 may provide a controlled, substantially constant force that presses the optical sensor 208 into the skin of the person 102, which may help the optical sensor 208 conform to curves and irregularities of anatomical structures along a chest region, for example.

The contact structure 1202 represents one example of a contact component configured to press the optical sensor 208 into the skin of the person 102. In other implementations, alternatively or in addition, the contact component may include one or more of a physical protrusion, a foam layer, or an inflatable balloon.

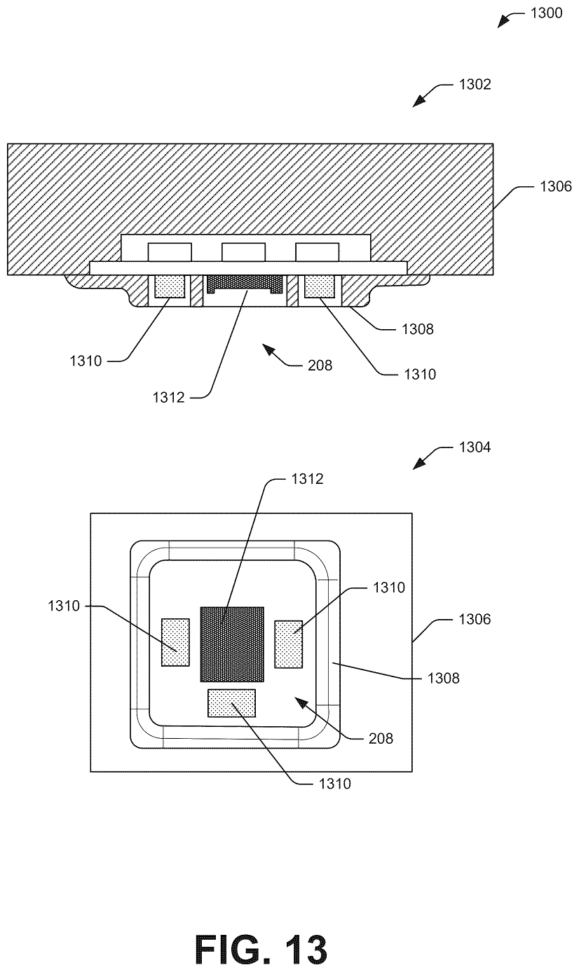

FIG. 13 depicts an example 1300 of an optical sensor configuration for the monitoring device 104. The example 1300 includes a side section view 1302 and a bottom view 1304. The side section view 1302 shows a cross-sectional representation of a sensor housing 1306 and associated components. A protrusion 1308 extends from a lower surface of the sensor housing 1306. The protrusion 1308 is configured to apply pressure against the skin of the person 102 when the monitoring device 104 is worn. By way of example, the protrusion 1308 may extend beyond a plane of a remaining portion of the monitoring device 104.

The optical sensor 208 is positioned within the protrusion 1308. The optical sensor 208 comprises at least one emitter 1310 and at least one photodetector 1312. The at least one emitter 1310 is configured to emit light toward the skin. The at least one emitter 1310 may include one or more light sources, such as light-emitting diodes (LEDs) and/or laser diodes. The at least one photodetector 1312 is configured to detect light reflected from the skin. The at least one photodetector 1312 may include one or more photodiodes, for example. As shown in the side section view 1302, barriers are positioned between the at least one emitter 1310 and the at least one photodetector 1312 within the protrusion 1308 to prevent light emitted by the at least one emitter 1310 from being directly detected by the at least one photodetector 1312.

In the example 1300, the optical sensor 208 includes three emitters 1310 and one photodetector 1312 positioned within the protrusion 1308, as particularly shown in the bottom view 1304. In other implementations, the optical sensor 208 may include any number of emitters 1310 and any number of photodetectors 1312 arranged in various configurations. The protrusion 1308 may force the optical sensor 208 into the skin, which may provide more consistent optical-to-skin contact and reduce likelihood of signal disruption with motion. The protrusion 1308 may also improve optical coupling by creating localized pressure at a sensing location.

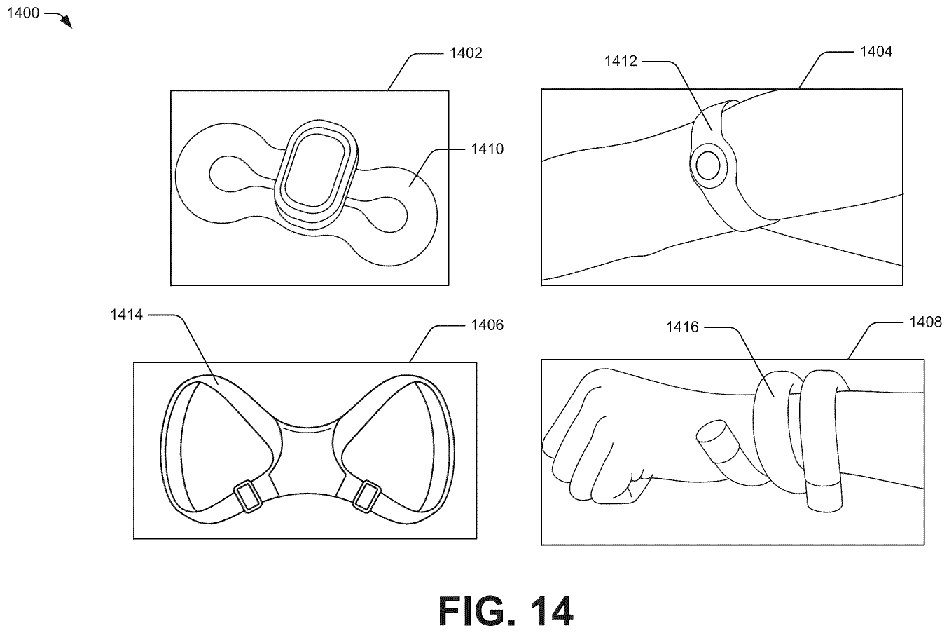

FIG. 14 depicts attachment method examples 1400 for the monitoring device 104. The attachment method examples 1400 include an adhesive attachment configuration 1402, a band attachment configuration 1404, a textile attachment configuration 1406, and a pressure/pinch attachment configuration 1408.

In the adhesive attachment configuration 1402, an adhesive patch 1410 is an attachment component configured to secure the monitoring device 104 and the optical sensor 208 to a chest region of the person 102, e.g., by adhering directly to the skin. The adhesive patch 1410, for example, may function similarly to the one or more adhesive portions 206 and/or the adhesive surface 506 described above.

In the band attachment configuration 1404, a band 1412 is an attachment component configured to wrap around a body part of the individual to secure the monitoring device 104. The band 1412 may have an adjustable and/or elastic configuration, with the optical sensor 208 on an inner surface of the band 1412 to maintain contact with the skin. The band 1412 may enable the monitoring device 104 to be affixed to the person 102 in a removable manner that does not use adhesive. The band 1412, for instance, is removable and repositionable.

In the textile attachment configuration 1406, a strap 1414 is an attachment component configured as a wearable harness to secure the monitoring device 104 to the individual. The strap 1414 may include adjustment mechanisms to allow for sizing adjustments. The strap 1414 may enable the monitoring device 104 to be removed and/or repositioned. It is to be appreciated that the textile attachment configuration 1406 may include additional or alternative textile-based attachment mechanisms, such as an adjustable elastic band configured to wrap around a body part (e.g., an arm or torso), a pocket integrated into a garment or armband configured to receive the monitoring device 104, hooks configured to interface with an existing strap (e.g., a fitness band), a sleeve, and/or the like.

In the pressure/pinch attachment configuration 1408, a pressure device 1416 is an attachment component configured to apply force on opposite sides of a body part (e.g., a wrist in this example) to hold the monitoring device 104 in place. In some implementations, the pressure/pinch attachment configuration 1408 may be configured for use on a torso of the person 102, such as via an over-shoulder mechanism or a torso-hugging mechanism. The pressure/pinch attachment configuration 1408 may help press the optical sensor 208 into the skin of the person 102. The pressure/pinch attachment configuration 1408 may be repositioned and/or reattached, which may enable use for extended monitoring periods.

The various attachment configurations shown in the attachment method examples 1400 may be selected based on desired attachment and performance characteristics of the monitoring device 104. Accordingly, in some examples, the monitoring device 104 is attachable to the chest region of the person 102 via one or more of an adhesive layer, a strap, a band, a textile, a pressure mechanism, a suction mechanism, a surface energy mechanism (e.g., using van der Waals forces and/or silicone substances), or another type of mechanism (e.g., a hand-held mechanism). Moreover, for each of the form factors discussed above, in various examples, the monitoring device 104 includes a self-contained “all-in-one” form factor, an encapsulated form factor, one or more auxiliary units, a satellite form factor configured to communicate with one or more additional devices, and/or the like.

In this way, the monitoring device 104 may be configured in a variety of form factors, including the example cartridge implementation 300, the example in-wing configuration 400, the example snap-in configuration 500, the example auxiliary configuration 600, and the example integrated optical sensor configuration 700. The optical sensor 208 may include the lens 518, which may have one or more of a convex configuration, a flat configuration, a protruding configuration, or a concave configuration. An adhesive component may include one or more of a sandwich configuration, a cartridge-compatible adhesive, an optically clear adhesive (OCA) bonded configuration, an optically clear resin configuration, an overlay, or an underlay. The monitoring device 104 may be attached to a chest region via one or more of an adhesive layer, a strap, a band, a textile, a pressure mechanism, a suction mechanism, a surface energy mechanism, or another type of mechanism. The contact structure 1202 or other contact components may be incorporated to maintain consistent contact between the optical sensor 208 and skin of the person 102. These various configurations and components may be combined and selected based on desired performance characteristics, attachment characteristics, and user comfort for wearing the monitoring device 104.

It should be understood that many variations are possible based on the disclosure herein. Although features and elements are described above in particular combinations, each feature or element is usable alone without the other features and elements or in various combinations with or without other features and elements.

What is claimed is:

1. A wearable device for physiological monitoring, comprising:
a housing configured to be attached to a chest region of an individual via an attachment component, the attachment component configured to conform to contours of the chest region; and
an optical sensor configured to detect a photoplethysmography (PPG) signal from the chest region of the individual.

2. The wearable device of claim 1, further comprising at least one of:
at least one electrode;
an accelerometer; or
a temperature sensor.

3. The wearable device of claim 1, wherein the housing includes a cartridge component that includes the optical sensor.

4. The wearable device of claim 1, wherein the attachment component is an adhesive component configured to surround a base of the housing and includes a plurality of petal regions extending radially away from the base.

5. The wearable device of claim 1, wherein the attachment component is an adhesive component including two or more wings, and the optical sensor is integrated into at least one wing of the two or more wings.

6. The wearable device of claim 1, wherein the housing is configured as a snap-in module that is removably attachable to a mounting interface that is coupled to the attachment component.

7. The wearable device of claim 1, further comprising a main body portion comprising one or more electrodes configured to detect an electrocardiography (ECG) measurement, and wherein the optical sensor is included in an auxiliary unit that extends from the main body portion.

8. The wearable device of claim 1, wherein the optical sensor includes a lens, the lens having one or more of a convex configuration, a flat configuration, a protruding configuration, or a concave configuration.

9. The wearable device of claim 1, further comprising a contact component configured to press the optical sensor into the chest region, the contact component including one or more of a spring, a foam layer, a physical protrusion, or an inflatable balloon.

10. The wearable device of claim 1, wherein the attachment component includes an optically clear adhesive configured to bond directly to skin of the individual to provide optical coupling between the optical sensor and the skin.

11. The wearable device of claim 1, wherein the attachment component includes one or more of an adhesive layer, a strap, a band, a textile, a pressure mechanism, a suction mechanism, or a surface energy mechanism.

12. A system for physiological monitoring of an individual, comprising:
a wearable monitoring device configured to be attached to a chest region of the individual, comprising:
a substrate having an adhesive disposed thereon configured to secure the wearable monitoring device to the chest region;
an optical sensor configured to detect a photoplethysmography (PPG) signal from the chest region of the individual;
a transmitter configured to transmit data based on the PPG signal; and
a housing configured to house at least the transmitter.

13. The system of claim 12, wherein the housing further houses the optical sensor, and wherein the adhesive includes a plurality of regions extending radially from the housing.

14. The system of claim 12, wherein the adhesive includes two or more wings extending from the housing, and the optical sensor is integrated into at least one wing of the two or more wings.

15. The system of claim 12, wherein the optical sensor is configured as a snap-in module that is removably attachable to a mounting interface coupled to the substrate.

16. The system of claim 12, wherein the wearable monitoring device is divided between a main body portion including the housing and an auxiliary portion including the optical sensor, the auxiliary portion being spaced away from the main body portion, and the system further comprises:
a flexible connecting element configured to electrically connect the auxiliary portion and the main body portion.

17. The system of claim 12, wherein the optical sensor includes at least one light emitter and at least one photodetector in a protrusion that is configured to press into skin of the individual when the wearable monitoring device is attached to the chest region.

18. A wearable monitoring device, comprising:
a substrate configured to be attached to a chest region of an individual via an adhesive component configured to conform to contours of the chest region;
one or more electrodes disposed on the substrate and configured to detect electrocardiography (ECG) measurements of the individual; and
an optical sensor attached to the substrate and configured to detect a photoplethysmography (PPG) signal from the chest region of the individual.

19. The wearable monitoring device of claim 18, wherein the optical sensor is housed in a snap-in module, and the wearable monitoring device further comprises a mounting interface affixed to the substrate, the mounting interface having one or more retention elements shaped to receive the snap-in module.

20. The wearable monitoring device of claim 18, further comprising:
an auxiliary portion spaced away from the substrate; and
a flexible connecting element coupled between the auxiliary portion and the substrate, wherein the optical sensor is disposed within the auxiliary portion and attached to the substrate via the flexible connecting element.
